(19) United States
(12) Patent Application Publication
(10) Pub. No.: US 2025/0339977 A1
(43) Pub. Date: Nov. 6, 2025

(54) ROBOTIC SYSTEMS WITH STERILITY ADAPTER AND RELATED METHODS

(71) Applicant: Monogram Technologies Inc., Austin, TX (US)

(72) Inventor: Gerardo Angel GONZALEZ CHAPA, Austin, TX (US)

(73) Assignee: Monogram Technologies Inc., Austin, TX (US)

(21) Appl. No.: 19/267,339

(22) Filed: Jul. 11, 2025

Related U.S. Application Data
(63) Continuation-in-part of application No. 18/773,996, filed on Jul. 16, 2024.
Application No. 18/773,996 is a continuation of application No. PCT/US2023/061119, filed on Jan. 23, 2023.
(63) Continuation-in-part of application No. 18/781,074, filed on Jul. 23, 2024.
Application No. 18/781,074 is a continuation of application No. PCT/US2023/061201, filed on Jan. 24, 2023.
(60) Provisional application No. 63/302,122, filed on Jan. 23, 2022.
(60) Provisional application No. 63/302,270, filed on Jan. 24, 2022.

(51) Int. Cl. B25J 15/04; A61B 17/00; A61B 34/32; B25J 11/00

(52) U.S. Cl. CPC B25J 15/0408; A61B 34/32; B25J 11/008; A61B 2017/00477

(57) ABSTRACT

Surgical robotic systems and methods that are configured to form and maintain a sterile field. The robotic systems include an articulated arm comprising a plurality of arm segments and adjustable joints, including a proximal base arm segment, a distal terminal arm segment and at least one intermediate arm segment extending therebetween. The robotic systems also include a sterility adapter comprising a first side removably coupled with the distal terminal arm segment, a second side and a surgical drape attachment interface portion. The robotic systems further include a surgical drape coupled to the surgical attachment interface portion and extending therefrom such that the surgical drape and the sterility adapter define a sterile field beneath the surgical drape and the second side of the sterility adapter. The robotic systems also include a robotic end effector removably coupled with the second side of the sterility adapter positioned within the sterile field.

### CROSS-REFERENCE TO RELATED APPLICATIONS

The present application is a continuation-in-part of U.S. patent application Ser. No. 18/773,996, filed on Jul. 16, 2024, and entitled Robotic Systems with Vibration Compensation, and Related Methods, published as U.S. Patent Publication No. US/2024/0366325 on Nov. 7, 2024, which is a bypass continuation of International Application No. PCT/US2023/061119, entitled Robotic Systems with Vibration Compensation, and Related Methods, filed on Jan. 23, 2023, published as PCT Publication No. WO/2023/141644 on Jul. 27, 2023, which perfects and claims priority benefit of U.S. Provisional Application No. 63/302,122, entitled Robotic Systems with Vibration Compensation, and Related Methods, filed on Jan. 23, 2022, the entireties of which are hereby expressly incorporated herein by reference. The present application is also a continuation-in-part of U.S. patent application Ser. No. 18/781,074, filed on Jul. 23, 2024, and entitled Active Robotic Systems with User Controller, published as U.S. Patent Publication No. US/2024/0423736 on Dec. 26, 2024, which is a bypass continuation of International Application No. PCT/US2023/141663, entitled Active Robotic Systems with User Controller, filed on Jan. 24, 2023, published as PCT Publication No. WO/2023/141644 on Jul. 27, 2023, which perfects and claims priority benefit of U.S. Provisional Application No. 63/302,270, entitled Active Robotic Systems with User Controller, filed on Jan. 24, 2022, the entireties of which are hereby expressly incorporated herein by reference.

### FIELD OF THE DISCLOSURE

The following disclosure relates generally to improved surgical robots, components thereof and related systems. More particularly, the following disclosure relates to surgical robotic systems with a sterility barrier adapter and a sterility barrier, components thereof and related systems that maintain a robust sterile barrier between nonsterile robot portions and sterile robot portions and a surgical field, enable rapid, intraoperative exchange of specialized end effector/tools with the robot, reduce surgical time and improves surgical efficiency while maintain a sterile field, and simplify sterilization workflows and reduces reprocessing time.

### BACKGROUND

A surgical robot is a computer-assisted device that enhances a surgeon's ability to perform complex procedures with high precision, flexibility, and control. These systems are used mainly in minimally invasive surgery (MIS), but are expanding into more fields due to advances in robotics, imaging, miniaturization, artificial intelligence (AIR) integration and increasing autonomy.

Surgical robots include a robotic arm, which is typically articulated, that either provides or facilitates the movement of the robot. End effectors of a surgical robot are the surgical instruments or tools attached to the tips or ends of the robotic arm. They are the parts that physically interact with the patient's body or otherwise aid in or perform surgical task, performing tasks like cutting, drilling, driving, grasping, suturing, cauterizing, or retracting tissue or holding, aligning or positioning a surgical implement. They are called “end effectors” because they sit at the “end” of the robotic arm and are often the final output point of the robot's motion. End effectors are typically designed and optimized for precision, dexterity, and/or to perform a specific surgical task, acting as the robot's (or a surgeon's) “hands”. Some end effectors are powered, while others are not.

The sterile field is a critical concept in surgery, including robotic procedures. It refers to the area that is kept free from microorganisms to prevent infection of a patient during an operation. In surgeries involving surgical robots, managing the sterile field requires special protocols because the robot introduces complex, multi-component systems into the operating room.

In robotic surgery, the sterile field includes the patient's surgical site, the surgeon's and other personnel's gloves and gown, an instrument table and tools, parts of the robot that interact directly or indirectly with the surgical site, and any area or components of the robot covered by sterile barrier or drapes that form or define a sterility barrier between the sterile and non-sterile fields/zones and components. The sterile barrier or drapes thereby define the sterile and non-sterile fields/zones and components.

Sterile draping (the process of covering surgical equipment, including robotic components, with sterile barriers (drapes or sheets) to maintain a sterile field and prevent infection during surgery) is therefore an important aspect of robotic surgery. Sterile draping may be especially important in robotic surgery because the robotic system is not inherently sterile and includes large components (like the arms/arm segments and end effector) that enter or hover over the sterile field (and may even interact with the surgical site/patient).

For example, before a surgery, a surgical team typically performs standard sterile prepping or sterilization of the patient and sterile field, including at least a distal portion of the robotic arm. Sterility barriers or drapes are then applied to the robotic arm and any parts that extend over or enter the sterile field. After draping, sterile surgical effectors/instruments are loaded onto the distal portion of the robot arm. The interfaces between the sterile tools and the robotic arm are carefully aligned without touching non-sterile areas.

The sterile drapes or barriers themselves are typically single-use light, flexible sterile sheets or covers that are continuous and prevent microorganisms from passing therethrough. They are commonly made from fluid-resistant, medical-grade sterile materials that are substantially impervious to microorganisms, fluids, and particles during surgery. Sterility barriers thereby prevent microorganisms on the non-sterile portions of a robot (such as on proximal portions of the robotic arm) from contaminating the sterile surgical site, and allow the robotic arm (such as the distal portion of the robotic arm with an end effector/instrument) to interact with sterile end effectors/instruments without breaching sterility.

Sterility barrier must stay intact and in place throughout a robotic surgery. If a sterility barrier tears or slips, it may require re-draping or restarting of the sterilization and preparation process. Improper draping, or sterility breaches, can lead to surgical site infections (SSIs), cross-contamination, and delays or surgery cancellations. Therefore, sterile draping is essential for patient safety and surgical success in robotic surgery.

The present disclosure provides improved surgical robotic systems with a sterility barrier adapter and a sterility barrier, components thereof and related systems that maintain a robust sterile barrier between nonsterile robot portions and sterile robot portions and a surgical field, enable rapid, intraoperative exchange of specialized end effector/tools with the robot, reduce surgical time and improves surgical efficiency while maintain a sterile field, and simplify sterilization workflows and reduces reprocessing time.

While certain aspects of conventional technologies have been discussed to facilitate disclosure of Applicant's inventions, the Applicant in no way disclaims these technical aspects, and it is contemplated that the inventions may encompass one or more conventional technical aspects.

In this disclosure, where a document, act or item of knowledge is referred to or discussed, this reference or discussion is not an admission that the document, act or item of knowledge or any combination thereof was, at the priority date, publicly available, known to the public, part of common general knowledge, or otherwise constitutes prior art under the applicable statutory provisions; or is known to be relevant to an attempt to solve any problem with which this specification is concerned.

### SUMMARY

The present inventions may address one or more of the problems and deficiencies of current surgical robots, surgical robot systems, components and related surgical methods. However, it is contemplated that the inventions may prove useful in addressing other problems and deficiencies in a number of technical areas. Therefore, the claimed invention(s) should not necessarily be construed as limited to addressing any of the particular problems or deficiencies discussed herein.

One problem solved by the surgical robots, surgical robot systems, components and related surgical methods of the present disclosure is enabling rapid, intraoperative exchange of robotic end effectors without compromising a sterile field vis a sterility adapter/interface. Without a sterile interface between the robot and an end effector or other surgical tool, removing or changing the end effector/tool would break sterility, introducing the risk of surgical site infection and requiring time-consuming re-draping/sterilization or workflow interruptions. Additionally, as robotic mounting points are nonsterile and cannot be autoclaved (or otherwise efficiently and/or effectively sterilized), making direct end effector/tool attachment unsafe within a sterile field. The surgical robots, surgical robot systems, components and related surgical methods of the present disclosure invention provides a sterile barrier adapter that allows for quick tool changes while preserving sterility, offers a reusable and easily sterilizable interface, and integrates with the surgical drape to maintain the sterile barrier around the robot arm. This improves surgical efficiency, reduces the risk of contamination, and supports the use of multiple specialized end effectors within a single procedure.

The present disclosure is generally directed to surgical robots, surgical robotic systems, surgical robotic system components and assemblies, and related methods (e.g., surgical methods and method of configuring a surgical robot or robotic system).

The present disclosure provides a surgical robotic system that forms a sterile field via, inter alia, a sterile or surgical drape or barrier, and that allows for a quick exchange of one end effector for another end effector on a robotic articulated arm in an intraoperative environment without compromising the sterile field. Because the end effector mounting portion of a robotic articulated arm (e.g., the mechanical and electrical interface at the terminal end portion of the robotic arm) cannot be sterilized and typically resides outside the sterile field, directly attaching and detaching end effectors/tools risks breaking sterility.

The present disclosure provides for rapid intraoperative exchange of robotic end effectors, allowing a user (such as a surgical team) to efficiently switch between different end effectors/tools, such as cutting or drilling tools, guides, jig, instruments or any other implement (e.g., a surgical or operative implement) depending on procedural needs. The present disclosure thus provides for preservation of the sterile field/barrier by utilizing a sterile barrier adapter/sterility adapter that interfaces between a nonsterile robot/robotic arm and a sterile end effectors so that end effector changes can be performed without violating sterility.

The present disclosure further provides for efficient sterilization workflow as the sterility adapter itself is designed as a removable quick-release component that can be easily detached and autoclaved between uses. The present disclosure also provides simple integration a sterile drape with a robotic system as the sterility adapter forms an intuitive mounting point for the sterile drape, further protecting the nonsterile robotic arm while maintaining easy access to the underlying sterile surgical field (beneath the sterile drape).

The systems, components and methods of the present disclosure thereby address a critical need for robotic systems in operating rooms or other sterile environments by combining rapid tool exchange with robust sterile barrier maintenance, improving both surgical efficiency and patient safety.

In one aspect, the present disclosure provides a surgical robotic system comprising an articulated arm, a sterility adapter, a surgical drape and a robotic end effector. The articulated arm comprises a plurality of arm segments and adjustable joints coupling adjacent arm segments of the plurality of arm segments that are configured to adjust the relative orientations of the adjacent arm segments. The plurality of arm segments comprises a proximal base arm segment, a distal terminal arm segment and at least one intermediate arm segment extending between the proximal base arm segment and the distal terminal arm segment. The sterility adapter comprises a first side removably coupled with the distal terminal arm segment, a second side and a surgical drape attachment interface portion. The surgical drape is coupled to the surgical attachment interface portion and extends therefrom such that the surgical drape and the sterility adapter define a sterile field beneath the surgical drape and the second side of the sterility adapter. The robotic end effector is removably coupled with the second side of the sterility adapter within the sterile field.

In some embodiments, the sterility adapter, the surgical drape and the robotic end effector are sterile. In some embodiments, the sterility adapter is autoclavable.

In some embodiments, the surgical drape attachment interface portion is positioned between the first side and the second side of the sterility adapter. In some embodiments, the surgical drape attachment interface portion extends about an axis of the sterility adapter, and wherein the surgical drape is coupled to the drape attachment interface portion about the axis of the sterility adapter such that the surgical drape extends about the sterility adapter and outwardly therefrom. In some embodiments, the surgical drape attachment interface portion comprises a ring portion with at least one flat surface coupling surface that extends about an axis of the sterility adapter. In some embodiments, the surgical robotic system further comprises a coupling member that removably couples the surgical drape and the attachment interface portion together.

In some embodiments, the robotic end effector is configured to facilitate a surgical procedure within the sterile field. In some embodiments, the robotic end effector is configured to interact with a surgical site of a patient within the sterile field. In some such embodiments, the robotic end effector is configured a cutting or drilling tool, a surgical guide or a surgical jig. In some other such embodiments, the robotic end effector is a powered end effector that effectuates movement of a cutting or drilling tool.

In some embodiments, the first side of the sterility adapter and an end portion of the distal terminal arm segment are configured as a mating quick-release coupling mechanism that releasably couples the sterility adapter and the distal terminal arm segment together. In some embodiments, the second side of the sterility adapter and a coupling portion of the end effector are configured as a mating quick-release coupling mechanism that releasably couples the sterility adapter and the distal terminal arm segment together. In some embodiments, the first side of the sterility adapter and an end portion of the distal terminal arm segment are configured as a first mating quick-release coupling mechanism that releasably couples the sterility adapter and the distal terminal arm segment together, and wherein the second side of the sterility adapter and a coupling portion of the end effector are configured as a second mating quick-release coupling mechanism that releasably couples the sterility adapter and the distal terminal arm segment together.

In another aspect, the present disclosure provides a method of configuring a surgical robot. The method comprises: removably coupling a first side of a sterile sterility adapter to a distal terminal arm segment of a robotic articulated arm; coupling a sterile surgical drape to a surgical attachment interface portion of the sterile sterility adapter such that the surgical drape extends therefrom and the surgical drape and the sterility adapter define a sterile field beneath the surgical drape and a second side of the sterility adapter; and removably coupling a first sterile robotic end effector to the second side of the sterility adapter within the sterile field.

In some embodiments, the method further comprises decoupling the first sterile robotic end effector from the second side of the sterility adapter while maintaining the sterile field, and removably coupling a second sterile robotic end effector to the second side of the sterility adapter within the sterile field while maintaining the sterile field.

In some embodiments, the method further comprises obtaining the sterile sterility adapter by autoclaving a sterility adapter.

In some embodiments, the surgical drape attachment interface portion extends about an axis of the sterility adapter, and wherein coupling the sterile surgical drape to the surgical attachment interface portion of the sterile sterility adapter such that the surgical drape extends therefrom comprises coupling the surgical drape to the drape attachment interface portion about the axis of the sterility adapter such that the surgical drape extends about the sterility adapter and outwardly therefrom.

In some embodiments, removably coupling the first side of the sterile sterility adapter to the distal terminal arm segment of the robotic articulated arm comprises mating a quick release coupling mechanism formed by the first side of the sterile sterility adapter and the distal terminal arm segment. In some embodiments, removably coupling the first sterile robotic end effector to the second side of the sterility adapter comprises mating a quick release coupling mechanism formed by a coupling portion of the first sterile robotic end effector and the second side of the sterile sterility adapter.

The surgical robotic systems, and methods of configuring a surgical robot, are advantageous as they enable rapid intraoperative end effector/tool changes on a surgical robot arm while maintaining a sterile field, which improves surgical efficiency and reduces the risk of contamination. The sterile barrier/sterility adapter, which may include dual quick-release mechanisms, the systems and methods allow seamless switching between specialized end effectors/tools without breaking sterility. Additionally, because the sterility adapter is designed to be sterilizable or single-use, it simplifies reprocessing between cases/uses and reduces turnaround time. The systems and methods improve workflow, enhance patient safety, and addresses a critical limitation of prior robotic systems which lack a sterile, efficient, and reusable end effector/tool interface.

The sterile barrier/sterility adapter of the present disclosure thereby interfaces between a nonsterile robotic arm and a sterile end effector. The sterility adapter may incorporate quick-release mechanisms on both sides-one side that connects to the robotic arm, and the other that connects to an end effector, thereby enabling rapid intraoperative end effector changes without compromising the sterile field. Such a dual quick-release design can support seamless switching between different end effectors, such as sagittal saws, rotary cutters, jigs or any other end effector or tool depending on needs/desires (e.g., surgical needs).

The sterile barrier/sterility adapter itself is designed to be sterile, and can be either autoclavable/reusable or manufactured (and packaged) as a single-use sterile product. The sterility adapter thereby provides flexibility in how sterility is maintained and ensures that a clean and sterile barrier is always present between the nonsterile robot portions and the sterile surgical field, significantly reducing the risk of contamination.

The sterile barrier/sterility adapter also allows a sterile surgical drape or barrier to be easily and securely mounted. For example, the sterility adapter includes an integrated feature or surface portion for drape attachment, such as a circumferential ring or interface surface (with at least one flat/planar side or surface portion). This coupling or junction between the surgical drape and the sterility adapter is configured to maintains the sterility of the sterile terminal portion of the robot arm (e.g., throughout a surgical procedure), while still permitting rapid end effector/tool exchange within the sterile field.

By combining removable couplings (e.g., a dual quick-release design), a sterile or sterilizable sterility adapter that combines/integrates with a surgical drape to create a sterile barrier and sterile field there-beneath, the disclose systems and methods provide an efficient, sterile, and safe solution for intraoperative effector/tool changes, improve surgical workflow, reduce infection risk, and overcome key limitations of current robotic systems.

It should be appreciated that all combinations of the foregoing aspects and additional concepts discussed in greater detail below (provided such concepts are not mutually inconsistent) are contemplated as being part of the inventive subject matter and to achieve the advantages disclosed herein.

These and other objects, features and advantages of this disclosure will become apparent from the following detailed description of the various aspects of the disclosure taken in conjunction with the accompanying drawings.

### BRIEF DESCRIPTION OF THE DRAWINGS

These and other features, aspects, and advantages of the present disclosure will become better understood when the following detailed description is read with reference to the accompanying drawings, which may or may not be drawn to scale, and in which like reference numerals represent like aspects throughout the drawings, wherein:

FIG. 1 illustrates an elevational perspective view of a portion of an exemplary surgical robotic system, in accordance with one or more aspects of the present disclosure.

FIG. 2 illustrates a side view of the surgical robotic system of FIG. 1, in accordance with one or more aspects of the present disclosure.

FIG. 3 illustrates an elevational perspective view of a distal terminal arm segment, a sterility adapter, an end effector and a surgical drape of the surgical robotic system of FIG. 1, in accordance with one or more aspects of the present disclosure.

FIG. 4 illustrates a side perspective view of the distal terminal arm segment, the sterility adapter and the end effector of the surgical robotic system of FIG. 1, in accordance with one or more aspects of the present disclosure.

FIG. 5 illustrates a side perspective view of the distal terminal arm segment, the sterility adapter, the end effector and the surgical drape of the surgical robotic system of FIG. 1, in accordance with one or more aspects of the present disclosure.

FIG. 6 illustrates an exploded elevational perspective view of a portion of the distal terminal arm segment, the sterility adapter, the end effector and a portion of the surgical drape of the surgical robotic system of FIG. 1, in accordance with one or more aspects of the present disclosure.

FIG. 7 illustrates an exploded side view of a portion of the distal terminal arm segment, the sterility adapter, the end effector and a portion of the surgical drape of the surgical robotic system of FIG. 1, in accordance with one or more aspects of the present disclosure.

FIG. 8 illustrates an elevational perspective view of a first side of the sterility adapter of the surgical drape of the surgical robotic system of FIG. 1, in accordance with one or more aspects of the present disclosure.

FIG. 9 illustrates an elevational perspective view of a second side of the sterility adapter of the surgical drape of the surgical robotic system of FIG. 1, in accordance with one or more aspects of the present disclosure.

### DETAILED DESCRIPTION

Aspects of the present disclosure and certain examples, features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the relevant details. It should be understood, however, that the detailed description and the specific examples, while indicating aspects of the disclosure, are given by way of illustration only, and are not by way of limitation. Various substitutions, modifications, additions, and/or arrangements, within the spirit and/or scope of the underlying inventive concepts will be apparent to those skilled in the art from this disclosure.

Approximating language, as used herein throughout disclosure, may be applied to modify any quantitative representation that could permissibly vary without resulting in a change in the basic function to which it is related. Accordingly, a value modified by a term or terms, such as “about” or “substantially,” is not limited to the precise value specified. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. In some instances, the approximating language may correspond to the precision of an instrument for measuring the value. Any examples of operating or configuration parameters are not exclusive of other parameters of the disclosed embodiments.

Terminology used herein is for the purpose of describing particular examples only and is not intended to be limiting. As used herein, the singular forms “a”, “an” and “the” are intended to include the plural forms as well, unless the context clearly indicates otherwise. Furthermore, references to “one example” are not intended to be interpreted as excluding the existence of additional examples that also incorporate the recited features. Moreover, unless explicitly stated to the contrary, the terms “comprising” (and any form of “comprise,” such as “comprises” and “comprising”), “have” (and any form of “have,” such as “has” and “having”), “include” (and any form of “include,” such as “includes” and “including”), and “contain” (and any form of “contain,” such as “contains” and “containing”) are used as open-ended linking verbs. As a result, any examples that “comprises,” “has,” “includes” or “contains” one or more step or element possesses such one or more step or element, but is not limited to possessing only such one or more step or element.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable or suitable. For example, in some circumstances, an event or capacity can be expected, while in other circumstances the event or capacity cannot occur—this distinction is captured by the terms “may” and “may be.”

The term “coupled” and like terms are used herein to refer to both direct and indirect connections. As used herein and unless otherwise indicated, the term “entirety” (and any other form of “entire”) means at least a substantial portion, such as at least 95% or at least 99%. The term “entirety” (and any other form of “entire”), as used herein, is thereby not limited to 100%, unless otherwise indicated. As used herein, the term “layer”

Components, aspects, features, configurations, arrangements, uses and the like described, illustrated or otherwise disclosed herein with respect to any particular embodiment may similarly be applied to any other embodiment disclosed herein.

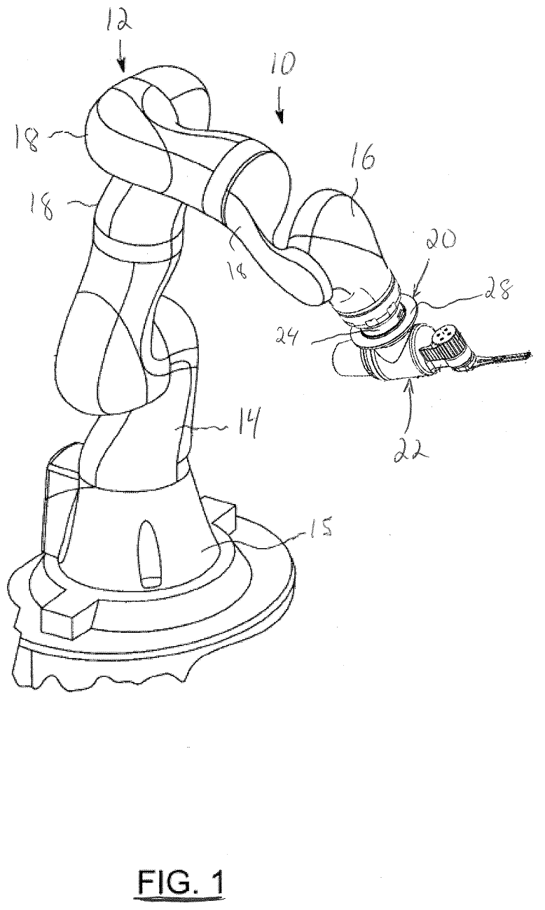

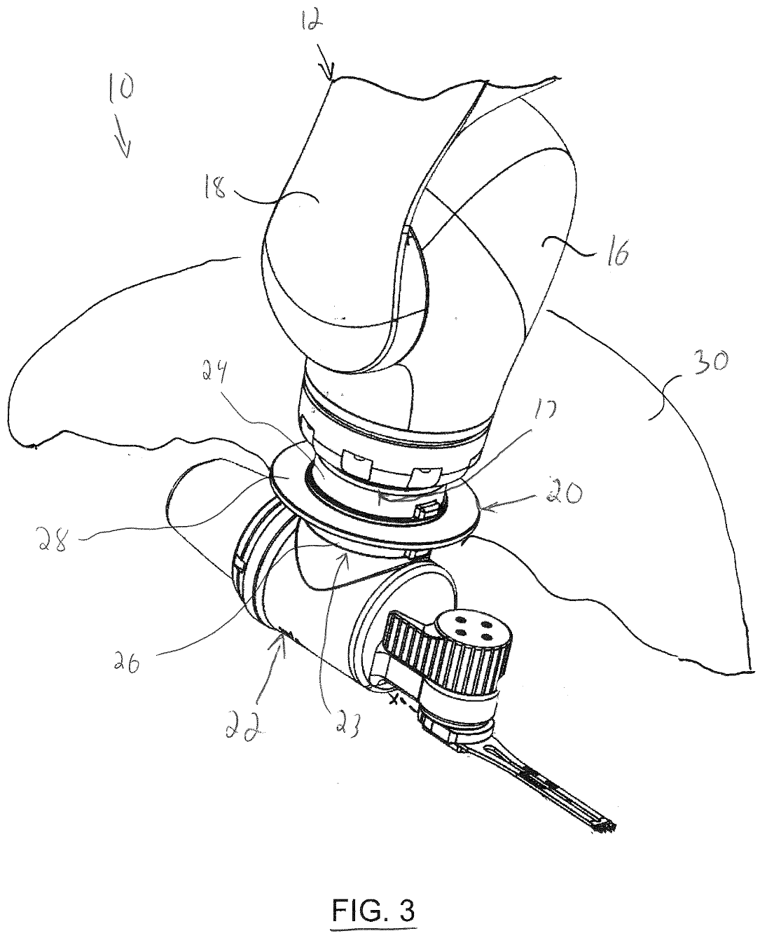

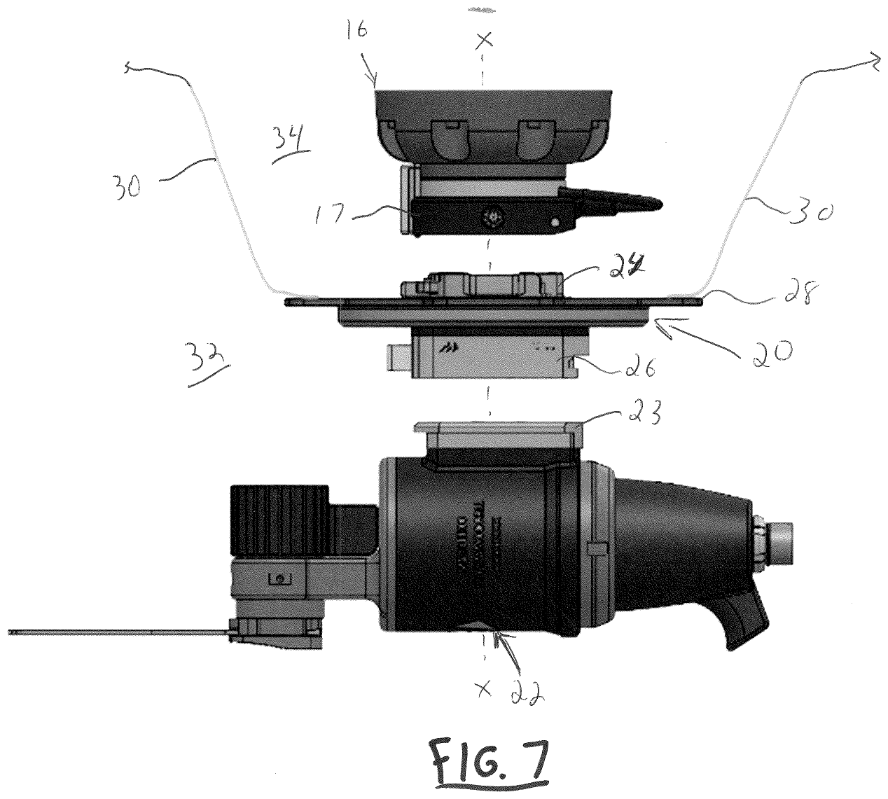

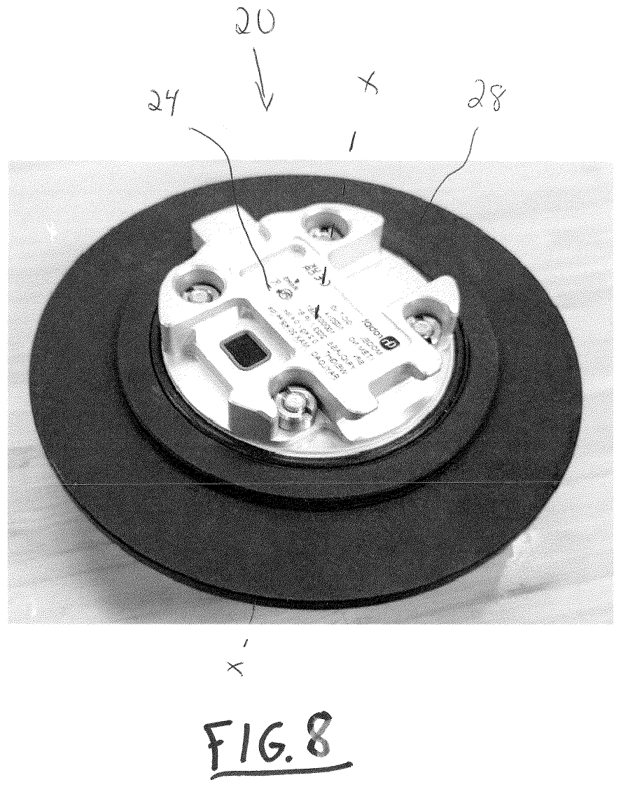

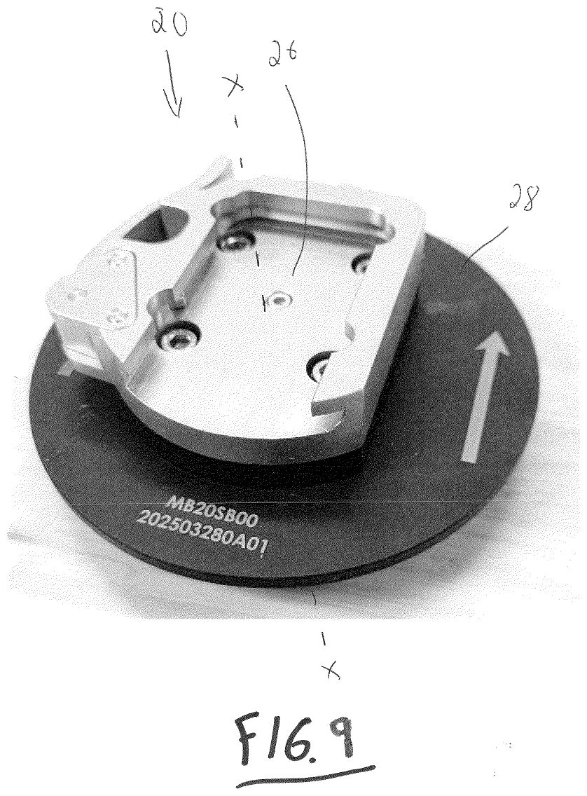

As shown in FIGS. 1-9, a robot or robotic system 10 with, inter alia, an articulated robotic arm 12, an end effector 22, a sterile barrier/sterility adapter 20 removably coupling the end effector 22 and the robotic arm 12, and a surgical or sterile drape or barrier 30 is disclosed. As depicted, the robotic system 10 may be configured as a surgical robot. For example, the surgical robotic system 10 may be biocompatible, and configured be sterilized to such a degree, as required in surgical settings. However, in other embodiments, the robotic system 10 may be configured as an industrial or other non-surgical robotic device or system. The term “surgical robot” as used herein in reference to the exemplary illustrative robot/robotic system 10 shown in FIGS. 1-10 (as well as to the exemplary illustrative robot/robotic system 110 shown in FIGS. 11-16) is not meant in a limiting sense, and any and all description herein directed to a “surgical robot” or “surgical robotic system” or the like equally applies to a generic robot/robotic system or an industrial or other non-surgical robot/robotic system.

As explained further below, the surgical robotic system 10 is advantageous as it maintains a robust sterile barrier between a nonsterile area portion 34 of the robotic system 10 and a sterile surgical field 32 within the sterile 32 surgical field, enables rapid, intraoperative exchange of specialized end effects or tools 22, reduces surgical time and improves efficiency, simplifies sterilization workflows and reduces reprocessing time, and/or provides compatibility with a wide range of end effectors 22.

The robotic system 10 may be operably connected to a computer system (e.g., memory, processor, etc.) (not shown) that controls movement of the end effector/tool 22, via movement of the articulated arm 12 for example, and potentially operation of the end effector 22. For example, in some embodiments, the robotic system 10 may comprise part of a robotic system that includes a control unit, and potentially a user interface (UI). The control unit may include at least one processing circuit, at least one input/output device, and at least one storage device or memory having at least one database or cutting instructions stored therein. The control unit may have a control algorithm or programming code for controlling the position of the end effector/tool 22 (such as via the joint angle between the segments 14, 16, 18 of the articulated arm 12, for example). The control algorithm or programming code may be a default control algorithm or include inputs from, for example, the UI and/or another interface.

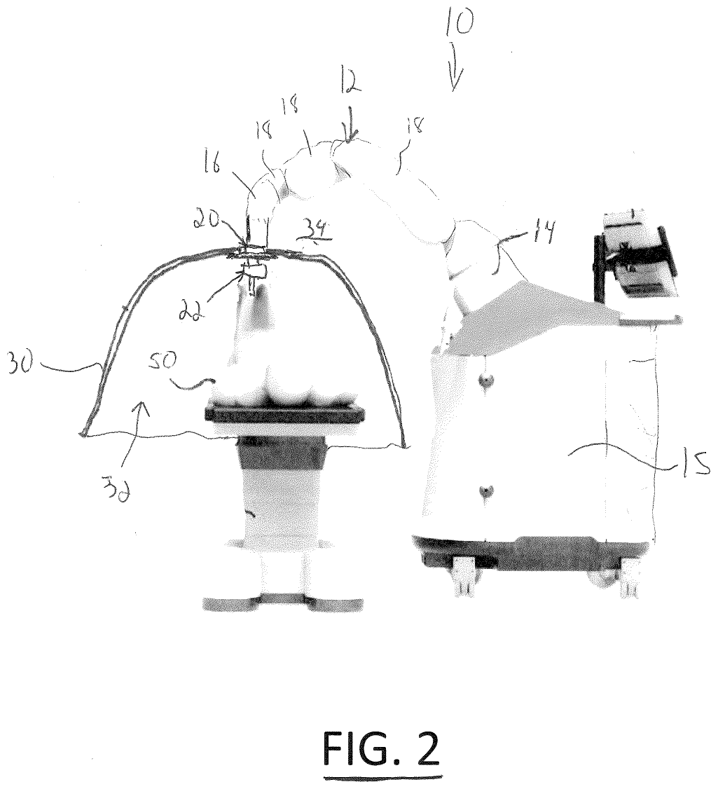

As shown in FIGS. 1 and 2, the articulated arm 12 may extend from a base 15 and include a plurality of rigid arm or body segments/parts, and a plurality of joints that connect adjacent segments. The plurality of joints may include, for example, four, five or six individual segments that are coupled together via three, four or five joints, respectively. In some other embodiments, the articulated arm 12 may include at least two segments and at least one joint coupling the at least two segments together, or more than six segments and more than five joints coupling the segments together. As shown, in the plurality of arm segments may comprises a proximal base arm segment 14 that is coupled to and extends from the base 15, a distal terminal arm segment 16 that defines the terminal distal end of the articulated arm 12 and is coupled with the sterility adapter 20 (as described further below), and one or more intermediate arm segments 18 extending between the proximal base arm segment 14 and the distal terminal arm segment 16.

Each arm segment 14, 16, 18 of the articulated arm 12 may define an axial axis extending along its longitudinal length. The joints between the arm segments 14, 16, 18 may be configured such that the arm segments 14, 16, 18 can rotate about their axes and/or articulate angularly with respect to each other such that the axes of adjacent segments 14, 16, 18 are angularly offset. In some embodiments, one or more of the joints may be configured to allow multiple degrees of freedom between adjacent arm segments 14, 16, 18 (and, potentially, the proximal base segment 14 and the base 15, and the distal terminal end segment 16 and the sterility adapter 20, for example). In some such embodiments, at least one of the joints may be configured to provide six degrees of freedom. The articulated arm 12 may further comprise motors, actuators or other adjustment devices that are configured to adjust the axial rotation and/or angular orientation between adjacent segments 14, 16, 18. In this way, the robotic system 10 can utilize the articulated arm 12 to translate an end effector 22 (which may include a tool) three-dimensionally in space and relative to a workpiece 50 (e.g., a patient) to, ultimately, perform or facilitate some sort of work or action on the workpiece 50. As noted above, the robotic system 10 may include control software that dictates or instructs, inter alai, the articulated arm 12 of the robotic system 10 to adjust in particular ways (i.e., adjustment of the joints) to accomplish prescribed movements of the end effector 22.

The base 15 of the surgical robotic system 10 may be fixed to or comprise, for example, a movable cart or the ground, such that the base 15 may provide a fixed frame of reference for defining the position, orientation, and motion of the plurality of joints and the plurality of arm segments 14, 16, 18 relative to the base 15. The base 15 may be used to define a frame of reference, such as, for example, a set of three-dimensional axes (e.g., x, y, z), which may be used to define positions, orientations, and motions of the surgical robotic system 10 and of objects relative to the surgical robotic system 10. A frame of reference defined relative to the base 15 may also be known as a world frame, a base, a base frame, a frame, or a tool frame. It is noted that with the position and orientation of an object defined or calculated in relation to the fixed frame of reference, the object may also be defined in the same frame of reference as the surgical robotic system 10, and the surgical robotic system 10 may calculate the position and orientation of the object. As such, the surgical robotic system 10 may programmably interact with the defined objects, positions, and/or orientations.

As shown in FIGS. 1-9, the sterility adapter 20 (and the end effector 22 via sterility adapter 20) may be rotatably coupled to the distal end, last or terminal/termination arm segment 16 of the articulated arm 12 via a rotatable connector portion of the distal terminal arm segment 16. The rotatable connector portion of the distal terminal arm segment 16 may be configured such that the sterility adapter 20 (and thereby the end effector 22) may be rotatable about an axis of the distal terminal arm segment 16 and an axis X-X of the sterility adapter 20.

The end effector 22 is the device, component, system, assembly, tool or combination thereof at the terminal end of a robotic arm 12, designed to interact with the environment or workpiece 50. The exact nature or configuration of end effector 22 may depend on the application of the robotic system, and numerous end effects 22 are known (e.g., there are many known surgical end effects designed to aid in surgery or surgical procedures). The end effector 22 may be any type or configuration of a robotic tool. In some embodiments, the end effector 22 is powered such that it comprises a motor or other device that effectuates movement, heat, light or other powered activity.

In some embodiments, the end effector 22 may include a tool, such as coupled via a chuck or the like. For example, the end effector 22 may be or include a surgical tool, and may be configured to facilitate a surgical procedure within a sterile field 32, or even interact with a surgical site of a patient 50 within the sterile field 32, as shown in FIG. 2. For example, in some embodiments, the end effector 22 may be configured as or include a cutting or drilling tool (such as a tissue cutting or drilling tool, such as tissue sagittal saw, tissue rotary cutter or drill, tissue drill or burr attachment, or the like), a surgical guide (e.g., such as a cutting guide or implementation guide) or a surgical jig (e.g., an alignment guide, or marking jig). In some such embodiments, the end effector 22 power a tool, such as a to effectuate movement (e.g., oscillation or rotation) of a cutting or drilling tool (e.g., a surgical tissue cutting or drilling tool).

In surgical embodiments, the robotic end effector 22 may be sterile. For example, the end effector 22 may be provided in a sterile state, such as in a sterile package, and attached to the sterility adapter 20 within an established sterile field 32 (beneath the sterile surgical drape 30 and the sterility adapter 20). In some embodiments, the robotic end effector 22 may be sterilized, or configured such that it can be sterilized, such as via an autoclave sterilization process (i.e., autoclavable).

The robotic system 10, as shown in FIGS. 1-9, allows for, and may include, a plurality of differing (sterile) end effectors 22 that are selectively utilized/coupled (e.g., one at a time) with the sterility adapter 20 within the sterile field 32. For example, one end effector 22 that is coupled with the sterility adapter 20 within the sterile field 32 may be decoupled from the sterility adapter 20 (and thereby from the robotic arm 12), and another different (sterile) end effector 22 may be coupled to/with the sterility adapter 20 within the sterile field 32 (and thereby to/with the robotic arm 12). As many (sterile) end effectors 22 as necessary or desired may hereby be used with the robotic system 10 without destroying or contaminated the sterile field 32. For example, the robotic system 10 (and namely the sterility adapter 20) thereby enables a user (e.g., a surgical team) to switch between (sterile) end effectors/tools 22 as needed for different stages of a procedure without interrupting sterility of the sterile field 32 (or workflow of the procedure and/or sterility).

As shown in FIGS. 1-9, the sterile barrier/sterility adapter 20 comprises a first coupling side or portion 24, a second coupling side or portion 26, a surgical drape attachment interface portion 28, and may define an axis X-X. The first coupling side 24 is configured to securely removably couple or mate to/with a coupling portion 17 of the distal terminal arm segment 16. In some embodiments, the first coupling side 24 of the sterility adapter 20 may removably couple to/with the distal terminal arm segment 16 such that the axis X-X of the sterility adapter 20 substantially aligns with an axis of the of the distal terminal arm segment 16. In some embodiments, the coupling portion 17 may be a rotatable or rotation distal portion of the distal terminal arm segment 16 that provides or allows for rotation thereof (or the sterility adapter 20) with respect to the proximal portion of the distal terminal arm segment 16.

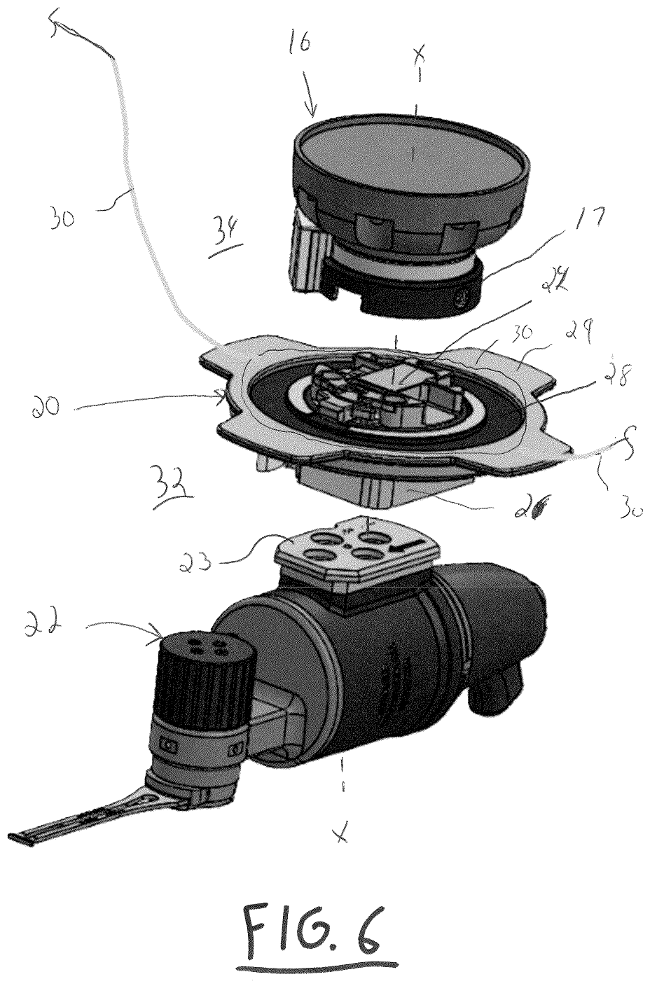

As shown in FIGS. 1-9, the first coupling side 24 of the sterility adapter 20 and the end or coupling portion 17 of the distal terminal arm segment 16 may be configured as a mating quick-release coupling mechanism that releasably couples the first coupling side 24 of the sterility adapter 20 and the coupling portion 17 of the distal terminal arm segment 16 together. For example, in some embodiments, as shown in FIGS. 6-8, the first coupling side 24 of the sterility adapter 20 may include at least one projection with outer undercut or peripheral groove, slot or recess. The sterility adapter 20 may also include a resilient member or detent that provides for resilient movement of a portion of the at least one projection or another portion or member of the first coupling side 24, such as with respect to the undercut or peripheral groove, slot or recess, that is configured to provide for selective or resistive decoupling of the first coupling side 24 of the sterility adapter 20 and the end or coupling portion 17 of the distal terminal arm segment 16. As also shown, the coupling portion 17 of the distal terminal arm segment 16 may include an opening, recess, slot, cavity or frame that is configured to accept or receive the at least one projection of the sterility adapter 20, and include at least one projection that extend into or mates with the outer undercut or peripheral groove, slot or recess of the at least one projection. In some other embodiments, the first coupling side 24 of the sterility adapter 20 and the coupling portion 17 of the distal terminal arm segment 16 may be conversely configured. It is also contemplated that the first coupling side 24 of the sterility adapter 20 and the coupling portion 17 of the distal terminal arm segment 16 may be otherwise configured to provide a mating quick-release coupling mechanism that releasably couples the sterility adapter 20 and the distal terminal arm segment 16 together. The first coupling side 24 (and the coupling portion 17) may thus be or form a mechanical latch or locking mechanism that securely attaches to the robotic arm while allowing rapid disconnection after a procedure.

The second coupling side 26 of the sterility adapter 20 is configured to securely removably couple or mate to/with a coupling portion 23 of the end effector(s) 22, as shown in FIGS. 1-9. In some embodiments, the coupling portion 23 may be a rotatable or rotation portion of the end effector 22 that provides or allows for rotation thereof (or the sterility adapter 20) with respect to a distal portion of the end effector 22. In some embodiments, the second coupling side 26 may be on or at (or form) an opposing side of the sterility adapter 20 as the first coupling side 24.

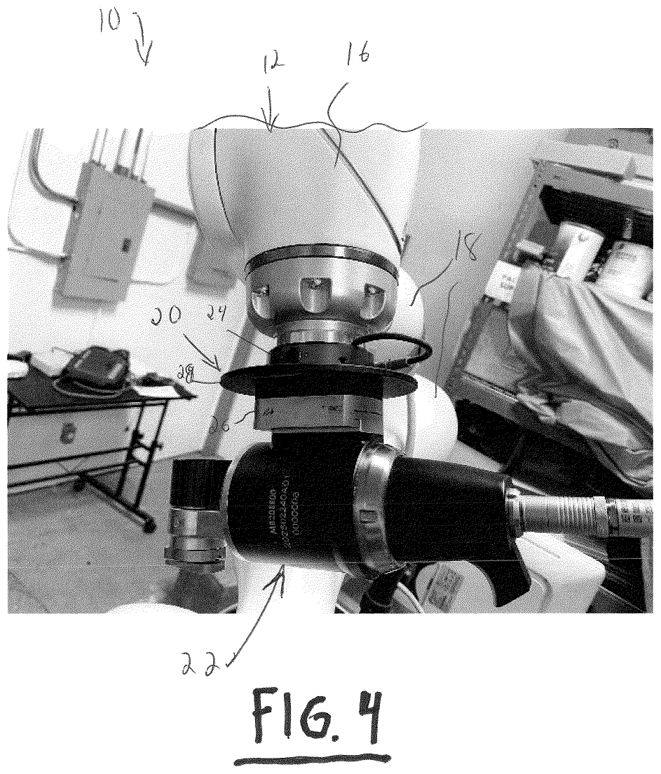

As shown in FIGS. 1-9, the second coupling side 26 of the sterility adapter 20 and the coupling portion 23 of the end effector(s) 22 may be configured as a mating quick-release coupling mechanism that releasably couples the second coupling side 26 of the sterility adapter 20 and the coupling portion 23 of the end effector(s) 22 together. For example, in some embodiments, as shown in FIGS. 4-7, the coupling portion 23 of the end effector 22 may include at least one projection with outer undercut or peripheral groove or recess, which may or may not be open at one side or peripheral end thereof. As shown in FIGS. 4-7 and 9, the second coupling side 26 of the sterility adapter 20 may include an opening, recess, cavity or frame that is configured to accept or receive the at least one projection of the effector 22, and include at least one projection that extends into or mates with the outer undercut or peripheral groove or recess of the at least one projection. The second coupling side 26 of the sterility adapter 20 may also include a resilient member or detent that provides for resilient movement of a portion of the second coupling side 26 that mates with the at least one projection or another portion or member of the end effector 22 that is configured to provide for selective or resistive decoupling of the second coupling side 26 of the sterility adapter 20 and the coupling portion 23 of the end effector 22. In some other embodiments, the second coupling side 26 of the sterility adapter 20 and the coupling portion 23 of the end effector 22 may be conversely configured. It is also contemplated that the second coupling side 26 of the sterility adapter 20 and the coupling portion 23 of the end effector 22 may be otherwise configured to provide a mating quick-release coupling mechanism that releasably couples the sterility adapter 20 and the end effector 22 together.

As shown in FIGS. 1-9, the surgical drape attachment interface portion 28 is configured to facilitate secure (and potentially substantially sterile) coupling, connection or mating of the surgical drape 30 and the sterility adapter 20. In some embodiments, the interface portion 28 may extend (e.g., continuously) about a periphery of the sterility adapter 20, such as extend about the axis X-X of the sterility adapter 20, as shown. For example, as shown in FIGS. 1-9, the surgical drape attachment interface portion 28 may comprise a ring portion with at least one flat surface coupling surface that extends about the axis X-X of the sterility adapter 20. As also shown, the interface portion 28 may be positioned between the first side 24 and the second side 26 of the sterility adapter 20.

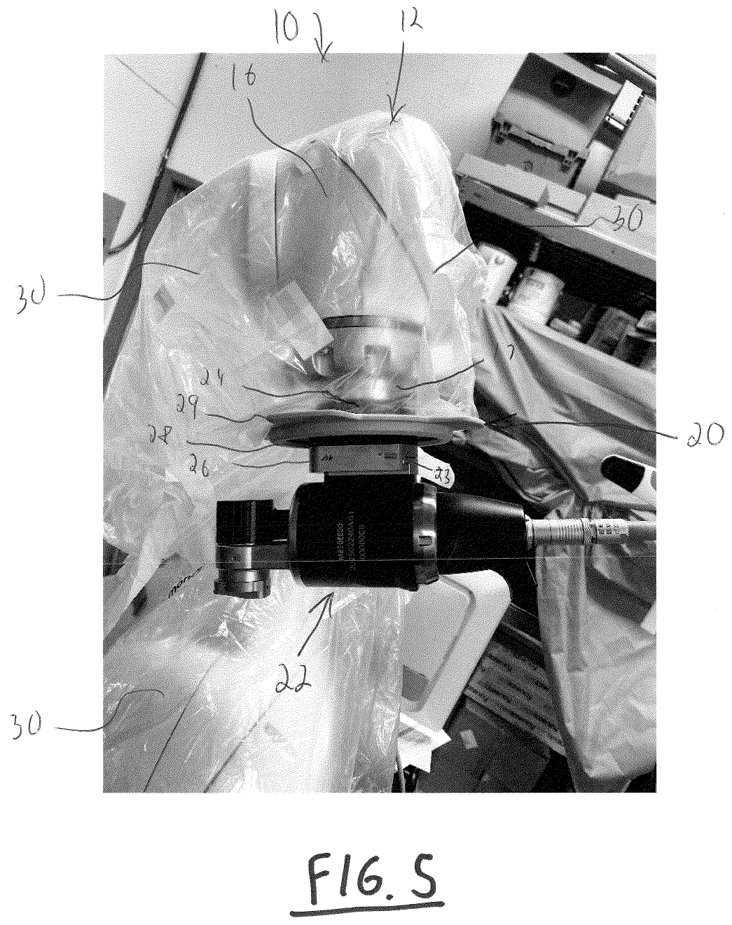

The surgical drape 30 may be coupled to the interface portion 28 via a variety of different mechanisms. For example, the surgical drape 30 may be coupled (such as removably coupled or fixedly coupled) with the interface portion 28 via one or more fasteners (e.g., touch fasteners, screws, etc.), clips, clamps, magnets, adhesives, resilient members, or the like, or via an interference or friction fit. For example, as shown in FIGS. 5 and 6, in some embodiments the robotic system 10 may include a coupling member 29 that coupled or fits on or over (e.g., via an interference, friction or resilient (e.g., stretched) manner) the interface portion 28 such that the coupling member 29 and the interface portion 28 are removably coupled. In such embodiments, the surgical drape 30 may be positioned between the coupling member 29 and the interface portion 28, or the surgical drape 30 may be affixed to the coupling member 29, such that that the coupling member 29 acts to couple the surgical drape 30 with the interface portion 28.

The sterility adapter 20 may be advantageously sterile. For example, the sterility adapter 20 may be provided in a sterile state, such as in a sterile package, and attached to the articulated arm 12 and/or the end effector 20 in a sterile state. In some embodiments, the sterility adapter 20 may be sterilized, or configured such that it can be sterilized, such as via an autoclave sterilization process (i.e., autoclavable). The sterility adapter 20 may thereby configured or designed to be sterile, and can be either autoclavable/reusable or manufactured (and packaged) as a single-use sterile adapter.

The sterility adapter 20 may prevent microorganism from passing therethrough. That is, the sterility adapter 20 may be configured to maintain sterility when coupled between the non-sterile area 34 and the sterile field 32, as shown. The sterility adapter 20 may thereby be a mechanical interface designed to connect the nonsterile robotic arm portions to the sterile surgical end effector 22 and surgical drape 30. The sterility adapter 20 may serve as a physical and sterile separation between the coupling portion 17 of the distal terminal arm segment 16 and the sterile end effector 22 and surgical drape 30.

The surgical drape 30 may be sterile, and be of any configuration that is effective in forming the sterile field beneath the surgical drape 30. For example, the surgical drape 30 may be a single-use (or reusable) light, flexible sterile sheet or coves that prevents microorganisms from passing therethrough. The surgical drape 30 may be made from fluid-resistant, medical-grade sterile materials that are substantially impervious to microorganisms, fluids, and particles during surgery. The surgical drape 30 thereby prevents microorganisms on the non-sterile portions 34 of a robotic system 10 (such as on proximal portions of the robotic arm) from contaminating the sterile surgical field 32 (and a surgical site, for example), and allow the robotic arm 12 (such as the distal portion 16/17) to interact with sterile end effectors 22 without breaching sterility.

As shown, in some embodiments, the surgical drape 30 is coupled to the surgical attachment interface portion 28 and extends therefrom such that the surgical drape 30 and the sterility adapter 20 define the sterile field 32 beneath the surgical drape 30 and the second side 28 of the sterility adapter 20. In some embodiments, the surgical drape 30 may be coupled to the drape attachment interface portion 28 about the axis X-X of the sterility adapter 20 such that the surgical drape 30 extends about the sterility adapter 20 and outwardly therefrom.

In use, prior to a surgery or other use, the sterility adapter 20 may be attached to the robotic arm 12 at the nonsterile mounting point 17 of the distal terminal arm segment 16, and may be and using a robot-side quick-release mechanism for example. The sterile surgical drape 30 may then fitted over the robotic arm 12 and attached to the drape attachment interface portion 28 of the sterility adapter 20, such as in a substantially secure and/or sterile manner.

During surgery or other use of the robotic system 10, sterile end effectors 20 can be quickly and securely attached and detached from the sterility adapter 20 (such as via a tool-side quick-release mechanism). This allows a user (e.g., a surgeon or surgical staff) to rapidly switch between instruments/tools/end effectors 22 without compromising the sterile field 32.

After a procedure or other use of the robotic system 10, the sterility adapter 20 may be removed from the robotic arm 12, and either sterilized (e.g., via an autoclave (if reusable)) or discarded (if single-use). The proximal portion of the robotic arm 12 itself may remain nonsterile and outside 34 the sterile field 32 at all times, avoiding the need for costly or impractical sterilization procedures on the robot hardware.

Many other examples will be apparent to those of skill in the art upon reviewing the above description. The scope of the various examples should, therefore, be determined with reference to the appended claims, along with the full scope of equivalents to which such claims are entitled.

While dimensions and types of materials may be described herein, they are intended to define parameters of some of the various examples, and they are by no means limiting to all examples and are merely exemplary.

In the appended claims, the terms “including” and “in which” are used as the plain-English equivalents of the respective terms “comprising” and “wherein.” Moreover, in the following claims, the terms “first,” “second,” and “third,” etc. are used merely as referee labels, and are not intended to impose numerical, structural or other requirements on their objects. Forms of term “based on” herein encompass relationships where an element is partially based on as well as relationships where an element is entirely based on. Forms of the term “defined” encompass relationships where an element is partially defined as well as relationships where an element is entirely defined. Further, the limitations of the following claims are not written in means-plus-function format and are not intended to be interpreted based on 35 U.S.C. § 112, sixth paragraph, unless and until such claim limitations expressly use the phrase “means for” followed by a statement of function cavity of further structure. It is to be understood that not necessarily all such objects or advantages described above may be achieved in accordance with any particular example. Thus, for example, those skilled in the art will recognize that the devices, systems and methods described herein may be embodied or carried out in a manner that achieves or optimizes one advantage or group of advantages as taught herein without necessarily achieving other objects or advantages as may be taught or suggested herein.

While the disclosure has been described in detail in connection with only a limited number of examples, it should be readily understood that the disclosure is not limited to such disclosed examples. Rather, this disclosure can be modified to incorporate any number of variations, alterations, substitutions or equivalent arrangements not heretofore described, but which are commensurate with the spirit and scope of the disclosure. Additionally, while various examples have been described, it is to be understood that aspects of the disclosure may include only one example or some of the described examples. Also, while some disclosure are described as having a certain number of elements, it will be understood that the examples can be practiced with less than or greater than the certain number of elements.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

1. A surgical robotic system, comprising:
an articulated arm comprising a plurality of arm segments and adjustable joints coupling adjacent arm segments of the plurality of arm segments that are configured to adjust the relative orientations of the adjacent arm segments, wherein the plurality of arm segments comprises a proximal base arm segment, a distal terminal arm segment and at least one intermediate arm segment extending between the proximal base arm segment and the distal terminal arm segment;
a sterility adapter comprising a first side removably coupled with the distal terminal arm segment, a second side and a surgical drape attachment interface portion;
a surgical drape coupled to the surgical attachment interface portion and extending therefrom such that the surgical drape and the sterility adapter define a sterile field beneath the surgical drape and the second side of the sterility adapter; and
a robotic end effector within the sterile field removably coupled with the second side of the sterility adapter.

2. The robotic system of claim 1, wherein the sterility adapter, the surgical drape and the robotic end effector are sterile.

3. The robotic system of claim 1, wherein the sterility adapter is autoclavable.

4. The robotic system of claim 1, wherein the surgical drape attachment interface portion is positioned between the first side and the second side of the sterility adapter.

5. The robotic system of claim 1, wherein the surgical drape attachment interface portion extends about an axis of the sterility adapter, and wherein the surgical drape is coupled to the drape attachment interface portion about the axis of the sterility adapter such that the surgical drape extends about the sterility adapter and outwardly therefrom.

6. The robotic system of claim 1, wherein the surgical drape attachment interface portion comprises a ring portion with at least one flat surface coupling surface that extends about an axis of the sterility adapter.

7. The robotic system of claim 1, further comprising a coupling member that removably couples the surgical drape and the attachment interface portion together.

8. The robotic system of claim 1, wherein the robotic end effector is configured to facilitate a surgical procedure within the sterile field.

9. The robotic system of claim 1, wherein the robotic end effector is configured to interact with a surgical site of a patient within the sterile field.

10. The robotic system of claim 9, wherein the robotic end effector is configured a cutting or drilling tool, a surgical guide or a surgical jig.

11. The robotic system of claim 9, wherein the robotic end effector is a powered end effector that effectuates movement of a cutting or drilling tool.

12. The robotic system of claim 1, wherein the first side of the sterility adapter and an end portion of the distal terminal arm segment are configured as a mating quick-release coupling mechanism that releasably couples the sterility adapter and the distal terminal arm segment together.

13. The robotic system of claim 1, wherein the second side of the sterility adapter and a coupling portion of the end effector are configured as a mating quick-release coupling mechanism that releasably couples the sterility adapter and the distal terminal arm segment together.

14. The robotic system of claim 1, wherein the first side of the sterility adapter and an end portion of the distal terminal arm segment are configured as a first mating quick-release coupling mechanism that releasably couples the sterility adapter and the distal terminal arm segment together, and wherein the second side of the sterility adapter and a coupling portion of the end effector are configured as a second mating quick-release coupling mechanism that releasably couples the sterility adapter and the distal terminal arm segment together.

15. A method of configuring a surgical robot, comprising:
removably coupling a first side of a sterile sterility adapter to a distal terminal arm segment of a robotic articulated arm;
coupling a sterile surgical drape to a surgical attachment interface portion of the sterile sterility adapter such that the surgical drape extends therefrom and the surgical drape and the sterility adapter define a sterile field beneath the surgical drape and a second side of the sterility adapter; and
removably coupling a first sterile robotic end effector to the second side of the sterility adapter within the sterile field.

16. The robotic system of claim 1, further comprising decoupling the first sterile robotic end effector from the second side of the sterility adapter while maintaining the sterile field, and removably coupling a second sterile robotic end effector to the second side of the sterility adapter within the sterile field while maintaining the sterile field.

17. The robotic system of claim 1, further comprising obtaining the sterile sterility adapter by autoclaving a sterility adapter.

18. The robotic system of claim 1, wherein the surgical drape attachment interface portion extends about an axis of the sterility adapter, and wherein coupling the sterile surgical drape to the surgical attachment interface portion of the sterile sterility adapter such that the surgical drape extends therefrom comprises coupling the surgical drape to the drape attachment interface portion about the axis of the sterility adapter such that the surgical drape extends about the sterility adapter and outwardly therefrom.

19. The robotic system of claim 1, wherein removably coupling the first side of the sterile sterility adapter to the distal terminal arm segment of the robotic articulated arm comprises mating a quick release coupling mechanism formed by the first side of the sterile sterility adapter and the distal terminal arm segment.

20. The robotic system of claim 1, wherein removably coupling the first sterile robotic end effector to the second side of the sterility adapter comprises mating a quick release coupling mechanism formed by a coupling portion of the first sterile robotic end effector and the second side of the sterile sterility adapter.
